(19) United States
(12) Patent Application Publication
(10) Pub. No.: US 2006/0122555 A1
(43) Pub. Date: Jun. 8, 2006

(54) Drug infusion device for neural axial and peripheral nerve tissue identification using exit pressure sensing

(76) Inventor: Mark Hochman, Lake Success, NY (US)

Correspondence Address: Anthony C. Kuhlmann, Ph.D.;Brown Rudnick Berlack Israels LLP, One Financial Center, Box IP, Boston, MA, 02111, US

(21) Appl. No.: 11/208,400

(22) Filed: Aug. 19, 2005

Related U.S. Application Data
(63) Continuation-in-part of application No. 10/827,969, filed on Apr. 20, 2004.
(63) Continuation-in-part of application No. 09/766,772, filed on Jan. 22, 2001, now Pat. No. 6,786,885.
Application No. 09/766,772 is a division of application No. 09/201,464, filed on Nov. 30, 1998, now Pat. No. 6,200,289.
(60) Provisional application No. 60/502,379, filed on Sep. 12, 2003.
(60) Provisional application No. 60/081,388, filed on Apr. 10, 1998.

(51) Int. Cl. A61M 31/00

(52) U.S. Cl. 604/67

(57) ABSTRACT

An automatic injection device includes a drive mechanism and a sensor used to determine an internal characteristic such as a force or internal pressure generated during an injection process. This characteristic is then used as a control parameter by a microprocessor or controller to determine the exit pressure of the fluid expelled by the device. This exit pressure is then used to identify the kind of tissue in which the injection is being introduced.

### RELATED APPLICATION DATA

This application is a continuation-in-part of U.S. application Ser. No. 10/827,969, filed Apr. 20, 2004, claiming priority to U.S. provisional application 60/502,379 filed Sep. 12, 2003.

This application is also a continuation-in-part application of U.S. application Ser. No. 09/766,772 filed Jan. 22, 2001, now U.S. Pat. No. 6,786,885, which is a division of application Ser. No. 09/201,464 filed Nov. 30, 1998, now U.S. Pat. No. 6,200,289, claiming priority to U.S. provisional application 60/081,388 filed Apr. 10, 1998.

### BACKGROUND OF THE INVENTION

1. Field of Invention

The present invention relates generally to improvements to the delivery of drugs, particularly to systems for subcutaneous injection/aspiration. More specifically this invention provides a method and device to the identification of specific tissue types (or soft-tissue density types) based on using a pressure measurement.

2. Description of the Prior Art

A regional anesthesia block of epidural tissue-space is understood to produce effective transient anesthesia of the lower extremities of the body. It can be effectively used for a vast number of invasive procedures of the body, including but not limited to child birth, prosthetic hip replacement and a variety of surgical procedures are necessary. It can also be effectively used as for treatment of chronic and acute pain management including, for example, “back-pain”, ailments of the vertebrae and, compression of the accessory nerves of the spinal colon. To achieve effective regional anesthesia and blockage of nerve transmission to the CNS the placement of an adequate volume of a local anesthetic solution must be deposited in close proximity to the spinal cord at a particular level of the vertebral column within the anatomic site known as the epidural “space”. The anatomic site is not a vacant “space” but actually an anatomic site containing a venous plexuses and fatty tissue which is continuous with the fat in the paravertebral space.

The epidural space is that part of the vertebral canal not occupied by the dura matter and its contents. It is a potential space that lies between the dura and the periosteum lining the inside of the vertebral canal. It extends from the foramen magnum to the sacral hiatus. The anterior and posterior nerve roots in their dural covering pass across this potential space to unite in the intervertebral bodies, and the intravertebral discs. Laterally, the epidural space is bordered by the periosteum of the vertebral pedicles, and the intervertebral foramina. Posteriorly, the bordering structures are the periosteum of the anterior surface of the laminae and articular processes and their connecting ligaments, the periosteum of the root of the spines, and the interlaminar spaces filled by the ligamentum flavum. The space contains venous plexuses and fatty tissue which is continuous with the fat in the paravertebral space.

The epidural tissue-space (posterior epidural space) is a limited anatomic area with an irregular shape measuring in several square millimeters with respect to cross section of the vertebrae and spinal column. The space is very narrow and is sometimes called a potential space, as the dura and the ligamentum flavum are usually closely adjacent. The space therefore has to be identified as the bevel of the needle exits the ligamentum flavum, as the dura will be penetrated shortly after if the needle penetrates any further. The standard technique to locating the epidural tissue-space is traditional employing a technique known as the “loss-of-resistance” technique. This technique utilizes a low-friction syringe made of plastic or glass connected to a epidural Touhly needle (16 to 18 gauge). Some use saline in the syringe and others use air.

The block can be performed with the patient either in the sitting or lateral decubitus position. The patient should be encouraged to adapt a curled up position, as this tends to open the spaces between the spinous processes and facilitates the identification of the intervertebral spaces. Epidurals can be sited at any level along the lumbar and thoracic spine, enabling its use in procedures ranging from thoracic surgery to lower limb procedures.

The clinician palpates the vertebral column at the appropriate level of the vertebral column between vertebrae. Local anesthesia is placed within the superficial tissues. A subcutaneous wheal at the midpoint between two adjacent vertebrae. The dermis is then punctured using the Touhly needle and the needle is advanced while the clinician simultaneously applies pressure on the plunger of the syringe. The pressure on the plunger will result in an amount of fluid continuously exiting out of the needle within the tissues unintentionally.

Insertion of the epidural needle continues and advanced through the supraspinous ligament, with the needle pointing in a slightly cephalad direction. Advancing the needle into the interspinous ligament, which is encountered at a depth of 2-3 cm. until the subjective sensation of increased resistance is felt as the needle passes into the ligamentum flavum. The needle is advanced until the subjective “feel” of resistance by the clinician results in a distinct “back-pressure” on the plunger. The clinician must subjectively differentiate the “back-pressure” or resistance encountered to identify the anatomic structure of the Ligamentum Flavum. The epidural space is entered by the tip of the needle after it passes through the ligamentum flavum.

It is known one of the deficiencies of this technique is when the tip of the needle is in the interspinous ligament there may be some loss of fluid into the tissues as the tissue is not particularly dense.

This movement of the Touhly needle from penetration of the dermis to identification of the Ligamentum Flavum can vary from greatly in depth depending on the patient's physical size. Overweight patients present a greater challenge because of the subjective nature of this technique, with the morbidly obese patient it may not be a suitable technique because of the limitations of subjective nature of this technique. Age appears to be an additional complicating factor that is difficult to assess because of the current subjective technical challenge because of the reduced size of the anatomy of the epidural tissue-space. Small child are therefore subject to the more dangerous procedure of general anesthesia when of the inadequacies of the subjective nature of this technique.

Unfortunately, if the Touhly needle moves once the epidural space has been located by either removal of the syringe or inadvertent movement of either the patient or doctors hand the needle can either be unknowingly outside the epidural tissue-space or worst advanced into dura of the spinal cord producing (production what is termed a “wet-tap”) which can have a dangerous long-term consequence to the patient. It is also possible that the space was initially properly located but once during the injection phase of depositing the anesthetic solution the needle advanced into the spinal cord depositing a bolus of anesthetic solution into the spinal cord resulting in transient or permanent damage to the patient.

Infusion pumps devices and systems are well known in the medical arts, for use in delivery or dispensing a prescribed medication to a patient and several attempts have been made to adapt these devices for the administration of an epidural injection and several attempts have been made to adapt these devices for the administration of an epidural injection.

Prior art references are known which attempt to utilize a pressure transducer to measure the pressure within the syringe (See for instance U.S. Pat. No. 5,295,967). A major deficiency of these systems is their inability to adjust the flow rate and/or pressure of the fluid to compensate for changes in resistances throughout the system, or to the exit pressure. (Exit pressure refers to the fluid pressure just downstream of the needle tip within the patient's body). Moreover, the prior art references fail to provide any means of determining this exit pressure.

U.S. Patent publication 2004/0215080 (application Ser. No. 10/481,527) and EP 0 538 259 discloses devices for locating an anatomic cavity that rely on an alarm (i.e. audible or visual warning signal) requiring the operator to manually modulate the drug delivery system during an injection procedure. These devices require subjective interpretation of events to which the operator must respond. Furthermore, these devices provide continuous injection fluid delivery and attempt to generate a sufficient pressure to do so via an automatic syringe pump device. This system does not, however, provide a means automatically controlling the injection pressure for fluid delivery or for aspiration of drug delivery during use. Thus, injection flow rate is maintained despite excess exit pressure that may result in pain and/or tissue damage.

The concept of using pressure as a metric to perform a safe and effective extradural injection (i.e. epidural injection) has been well documented in the medical literature. Pressure has been used to identify the epidural space and the importance of pressure within the epidural space has been described by a variety of researchers over the years utilizing a variety of experimental set-ups. Usubiaga and co-workers discussed the relationship of pressure and the epidural space while performing an injection into the epidural space and tissues (Anesth. Analg., 46: 440-446, 1967). Husenmeyer and White described the lumber extradural injection technique and relationship of pressure of during injection in pregnant patients (Br. J. Anaesth., 52: 55-59, 1980). Other investigators, including Paul and Wildsmith (Br. J. Anaesth., 62:368-372, 1989) and Hirabayashi et al. (Br. J. Anaesth., 1990 65:508-513), also evaluated the relationships between pressure and resistance of their effects on the administration of an epidural injection. And, Lakshmi Vas and co-workers have extended these principles into the area of pediatric medicine (Pediatric. Anesth. 11:575-583, 2001).

Lechner and co-workers described a system for epidural injections (Anesthesia, 57:768-772, 2002; Anesth. Analg. 96:1183-1187, 2002; Euro. J. Anaestheol. 21:694-699, 2004). This system improves the reliability of epidural injection administration by providing an audible and/or visual signal when the system detects the loss of resistance associated with needle entry into the epidural space.

U.S. Pat. No. 6,200,289, a parent of the present application and incorporated herein by reference, discloses an automatic injection device that includes a drive mechanism that causes a therapeutic fluid to flow from a cartridge supported by a cartridge holder, a tube and a handle with an injection needle. The drive mechanism is connected to an electric motor and a sensor positioned at the motor output that measures the force applied by the motor to the drive mechanism. This force is then used to determine an internal characteristic such as a force or internal pressure generated during the injection process. This characteristic is then used as a control parameter by a microprocessor or controller which generates corresponding commands to the drive mechanism. In a particularly advantageous embodiment, the characteristic is used to calculate an exit pressure at which fluid ejected by the device through an elongated tube. The electric motor, is then operated in such a manner that the exit pressure is maintained at a predetermined level to insure that a patient does not suffer pain and/or tissue damage.

### SUMMARY OF THE INVENTION

The present invention provides a method and device that enables the practitioner to accurately and reproducibly administer an injection to a patient in a desired tissue location. The device and method limit the amount of pain and tissue damage associated with the injection, the risk of complication from a misplaced injection, and also reduces the amount of injection fluid that is administered to non-target tissues. The current device utilizes the inherent tissue density or resistance of fluid pressure (exit pressure) within that tissue to identify the accuracy of placement of a needle within specific tissues. Each tissue has its own pressure density characteristics which are represented as measurable pressures that can be elicited within a given tissue type. The density or resistance of the tissue is measured using the exit pressure of a fluid infused from a computer-controlled drug delivery system capable of detecting pressure resistance during infusion. Based on the known tissue densities of the injection target tissue and the surrounding non-target tissue, the practitioner may select and pre-set a maximum exit pressure value. During injection, the device automatically limits the flow rate of the injection fluid such that the pre-set maximum exit pressure is never exceeded. Thus, under injection conditions where the exit pressure measurement exceeds the maximum pre-set exit pressure, the injection fluid flow rate is reduced to zero.

Thus, an injection device of this invention includes a fluid reservoir (fluid storage device), an injection fluid, a pumping mechanism, an end in fluid contact with the reservoir and adapted to be inserted into the body of a patient, a sensor arranged to determine a resistance measurement of the injection fluid, and a controller capable of receiving the resistance measurement from the sensor, calculate an exit pressure, and modulating the flow rate of the injection fluid. The sensor may be an in-line sensor placed between the pumping mechanism and the end, but is preferably between the pumping mechanism and the beginning of the tubing set which measures the pressure of the injection fluid. Alternatively, the sensor may be within the mechanical arm.

A sensor, such as a transducer, is used to sense the force or pressure generated by the motor and applied by the plunger within the fluid storage device. In one aspect of the invention, the transducer measures the force between the carpule adapter and the remaining housing of the device. In another aspect of the invention, the transducer includes a size sensing device that senses a change in dimension of an element of the device, said change being indicative of the force or pressure of the drug within the system and the exit pressure. For example, the change in size of the tubing may be used as an indicia of this force or pressure. In another embodiment, the pressure within the tube is measured externally and used as a means of determining the exit pressure.

It is contemplated that the controller is capable of accepting user-inputted parameters including, for example, a pre-set maximum exit pressure and a pre-set maximum flow rate, as well as information about the injection apparatus including, for example, tubing material, length, and bore, injection fluid temperature, viscosity, and composition. The controller is further capable of modulating the flow rate, including reducing the flow rate to substantially zero, to maintain the measured exit pressure at a value less than the pre-set maximum exit pressure. The flow rate may be controlled in a binary manner (i.e., at a pre-set flow rate when the measured exit pressure is less than the pre-set maximum exit pressure, and off when the measured exit pressure is less than the pre-set maximum exit pressure), or the flow rate may be a function of the exit pressure (i.e., the flow rate is higher at measured exit pressures farther below the pre-set maximum exit pressure). In the latter case, the flow rate may, optionally, be preset to a maximum allowable flow rate. Likewise, the function relating the flow rate to the measured exit pressure may also be user-defined. In useful embodiments, the pre-set maximum exit pressure is between about 50 mm/Hg and about 300 mm/Hg, or between about 150 mm/Hg and about 250 mm/Hg.

The pressure resistance measure is optionally converted into a visual as well as audible signal on a continuous basis. The measurements are then presented to the doctor so that the doctor can determine or confirm whether the injection is being delivered to the right tissues. In addition, the measurements are also recorded for later review and documentation of the clinical event. Upper limits of pressure as well as control of flow-rate can be pre-defined to ensure that excessive pressure and/or flow-rate are not used during this process.

The invention, therefore, provides a method for administering an injection to a patient by providing a fluid reservoir, an injection fluid, a pumping mechanism, and an end adapted for insertion into the patient; pumping the fluid from the reservoir into the patient; calculating the exit pressure of the fluid at an interface between the end and the tissue of said patient, and controlling the flow rate of the injection fluid such that the exit pressure does not exceed a pre-set maximum exit pressure.

In one embodiment, the devices and methods of this invention are used to administer an epidural injection. In this embodiment, the injection fluid contains, for example, an anesthetic and the end is adapted for insertion into the epidural tissue space. It is contemplated that either the pharmaceutical-containing or a pharmaceutical-free (testing) fluid is used to identify the epidural tissue space during the needle placement phase of the epidural procedure. Suitable pharmaceutical-free fluids include, for example, physiological saline, phosphate-buffered saline, artificial cerebral spinal fluid, Ringers, 5% dextrose, or filtered air. Once the epidural tissue space is identified using the loss-of-resistance method, the injection fluid is changed (i.e., requiring a plurality of fluid reservoirs) to a pharmaceutical-containing fluid. The use of a pharmaceutical-free fluid during the needle placement phase minimizes or eliminates the delivery of the pharmaceutical to non-target tissues.

Frequently, procedures that require an epidural injection of anesthetic are lengthy and, in addition to the initial (loading) dose, one or more subsequent (maintenance) doses are required. Typically, an indwelling catheter is used to administered the plurality of doses. In another embodiment, the invention provides a method for administering an epidural injection requiring a plurality of injections wherein, during administration of the second (and subsequent) doses, the exit pressure of the fluid at an interface between the end and the tissue of said patient is calculated, and the flow rate of the injection fluid during said second injection is controlled such that the exit pressure does not exceed the pre-set maximum exit pressure. Likewise, this technique may be used for indwelling catheter maintenance (i.e., to determine whether the catheter remains in a target tissue such as the epidural tissue space) whether or not an additional injection is contemplated or desired at that time.

It is further contemplated that this injection device may be used for aspiration during an epidural procedure. Aspiration may be used either to withdraw a sample of tissue or extracellular fluid (i.e., cerebral spinal fluid), or may be used to determine the correct placement of the injection needle. During an aspiration procedure, the “entry pressure” is measured in the same manner as the exit pressure with the epidural tissue space characterized by a loss-of-resistance. Likewise, false loss-of-resistance is also identified using an aspiration procedure because the internal tissue structure (i.e., cyst) will be quickly drained of its contents and the entry pressure will rise above the threshold entry pressure.

The present application also provides alternate means of determining force or pressure within an automatic injection device. In one embodiment, the electrical energy or power used by the motor is used as a parameter indicative of the force. In another embodiment, a change in a dimension of various elements of the fluid delivery system are used as parameters. This dimensional change is then converted into signal indicative of the internal force/pressure. For example, some of the elements that exhibit dimensional changes responsive to increased internal forces or pressures include the cartridge or reservoir holder, including its wings, the tube used to deliver the drug from the cartridge to the handpiece, the needle hub and/or its elements. The sensor for determining this dimensional variation may be for example an optical sensor.

Another method is to determine the stress or strain on the motor housing and/or the supporting members of the drive. A standard electronic strain gauge may be used for making this measurement.

The motor, the coupling associated with the motor and the electronic controller discussed below is at least partially disposed within the housing for protection.

The fluid storage device is filled and a setup process is initiated during which various operational parameters are calculated, retrieved or received from the clinician. The clinician also specifies the fluid flow rates and peak exit pressure and a total amount of fluid to be dispensed. Then he operates a pneumatic control such as a foot pedal and initiates the fluid flow. Alternatively, commands may be initiated by the clinician either electronically or by voice commands. During dispensing, the output from the transducer is used to calculate the current exit fluid pressure. If this exit pressure approaches a certain threshold, the fluid flow rate is automatically reduced to prevent excessive exit pressure, thereby ensuring that the patient does not suffer undue pain and no tissue is damaged. Several optional features are also provided including aspiration, purging or charging the media with or without air.

Throughout the process, the clinician is provided with constant current information on the ongoing process, both visual and aurally, including the current flow rate, total volume ejected or aspired, exit or entry pressures and other parameters. The slave microprocessor receives commands from the master microprocessor and generates the drive signals required to operate the motor.

### BRIEF DESCRIPTION OF THE DRAWINGS

FIG. 1 shows a diagram illustrating the major components of the infusion device constructed in accordance with this invention;

FIG. 2 shows an orthogonal view of the drive mechanism of FIG. 1;

FIG. 3 shows internal details of the drive mechanism of FIG. 1;

FIG. 3A shows a block diagram of the electronic controller of FIG. 1;

FIG. 4 shows a side view of the housing of a different type of infusion device with an adapter for pressure sensing;

FIG. 5 shows an end view of the housing of FIG. 4;

FIG. 6 shows an enlarged view of the adapter of FIG. 4;

FIG. 7 shows a somewhat diagrammatic cross-sectional view of the housing of FIG. 4;

FIG. 8 shows an alternate embodiment of a pressure gauge using the size of the tubing;

FIG. 9 shows another embodiment of the pressure gauge using the size of the tubing;

FIG. 10 shows a graph of typical pressure ranges for injections into four different types of tissues.

FIG. 11 is a photograph of an injection apparatus configured in accordance with the principles of this disclosure.

FIG. 12 is a graph of exit pressure (mm/Hg) in tissues normally encountered during administration of an epidural injection.

FIG. 13 is a graph of exit pressure (mm/Hg) during an epidural injection where t=0 is prior to the catheter puncturing the skin.

### DETAILED DESCRIPTION OF THE INVENTION

The subject invention pertains to a system for delivering drugs such as an anesthetic, under pressure into a patient's tissues. Importantly, due to a variety of factors, injected fluid disperses through a tissue at different rates, causing the fluid exit pressure to vary. The present inventor has discovered that this exit pressure (or an internal pressure related to the exit pressure) is indicative of, and may be used to identify several types of tissues.

The present invention provides a method and device that enables the practitioner to accurately identify central or peripheral nervous tissues and associated structures (i.e. the epidural space, extradural space) and perform a diagnostic and therapeutic procedure. The current device utilizes the exit pressure of a fluid from a needle or catheter (“the injector”) following placement of the needle/catheter within the tissue in order to properly identify the accuracy of placement and to monitor the (correct) placement during an injection or aspiration. Specifically, the present device utilizes exit pressure to identify accurate needle/catheter placement throughout the insertion, injection, and maintenance phases of the procedure. First, the exit pressure is used during the needle/catheter insertion to identify the subcutaneous anatomical structures and to enable the clinician to correctly determine when the lumen of the injector is placed within the epidural tissue space. The exit pressure is also used during the maintenance phase of the procedure to ensure that the injector remains in the epidural tissue space. The is particular risk during medical procedures that require an initial epidural injection (i.e., loading dose) followed by periodic maintenance doses in order to maintain the desired level of anesthesia. Typically, an indwelling catheter is inserted into the epidural space and remains attached to the injection device throughout the procedure. Frequently, the patient is moved between the loading dose and one or more of the maintenance doses. Such movement may cause a correctly placed catheter to be migrate from the epidural tissue space into a non-target tissue. The present device monitors the exit pressure during all periodic doses (i.e., the loading dose and all subsequent maintenance doses). Thus, the clinician is alerted should the catheter migrate during the maintenance phase. procedure after a catheter has been inserted, and an initial loading dose of anesthetic administered, but prior to the administration of the final main In addition, the current device utilizes exit pressure to properly identify the accurate placement of an indwelling catheter.

Thus, the advantages of the present device over the prior art include (i) a means to identify the epidural tissue space while utilizing a negligible volume of drug-containing solution, (ii) a means to identify a “false-positive” (i.e., a cyst, cavity, or other area of low resistance that is not the epidural space), (iii) a means to mitigate the risk of puncturing the spinal cord by advancing a catheter or needle through the epidural space (i.e., producing a wet tap) during the administration of an epidural injection, and (iv) a means to monitor the placement of a catheter for the entire duration of catheterization (i.e., during the maintenance phase of drug infusion).

According to the principles of this disclosure, the exit pressure is measured using the pressure/force of a fluid injected/infused from a computer-controlled drug delivery system capable of detecting pressure resistance during infusion. The pressure resistance measure is converted into a visual as well as audible signal on a continuous basis. The computer-controlled drug delivery system is continuously modulated based on the exit pressure generated. Thus, the flow-rate is variable and is dependent on the exit pressure of the system. It is contemplated that the exit pressure is the primary controlling variable of the system. The flow-rate, therefore, becomes a secondary variable that is modulated within a defined range in order to maintain the desired exit pressure. In one specific embodiment, the fluid flow is stopped at exit pressures exceeding a pre-determined threshold (maximum exit pressure). The flow-rate, as a secondary variable, may be limited so that fluid injections are not unduly rapid under low pressure conditions. It is contemplated that the relationship between exit pressure and fluid flow rate may either be binary or continuous. A binary relationship exists when the injection device is configured to deliver a fluid at a single, pre-determined flow rate for any exit pressure less than the pre-set maximum. Thus, the fluid flow is either on or off based on whether or not the exit pressure exceeds the threshold. Alternatively, the flow rate may be modulated by as a function of exit pressure. In this case, flow rate will be reduced as the maximum exit pressure is approached and increased as the exit pressure drops. Optionally, the flow rate may be limited to a pre-set maximum. This is a desirable configuration when injections are made into high resistance tissues and “fluid pulsing” is undesirable.

It is also contemplated that the injection device optionally may contain a means for recording and/or displaying relevant injection data including, for example, instantaneous flow rates, exit pressures, and injection amounts. All measurements and information may be presented to the clinician in “real-time” so that the clinician may determine whether the injection is being delivered to the intended location and/or correct tissues and may modify the injection technique accordingly. In addition, the measurements may be recorded for later review and documentation of the clinical event.

Exit Pressure-Controlled Injection Device

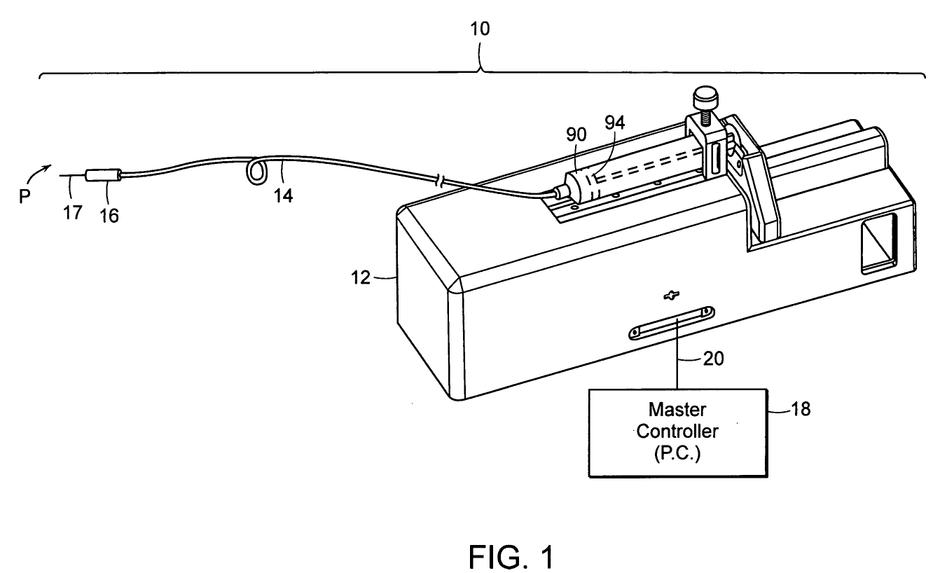

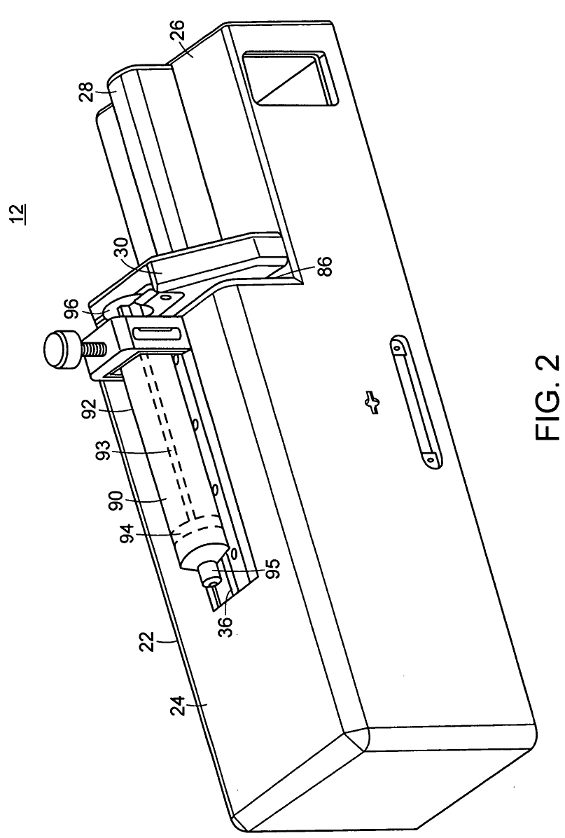

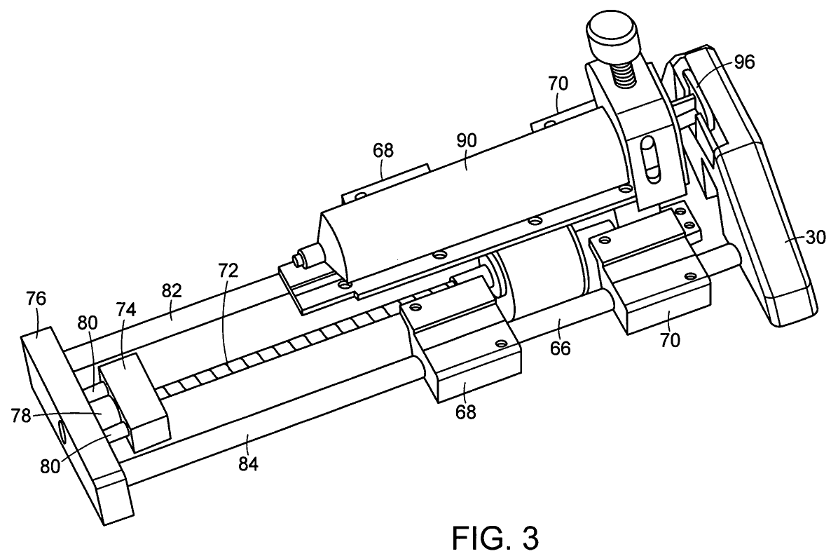

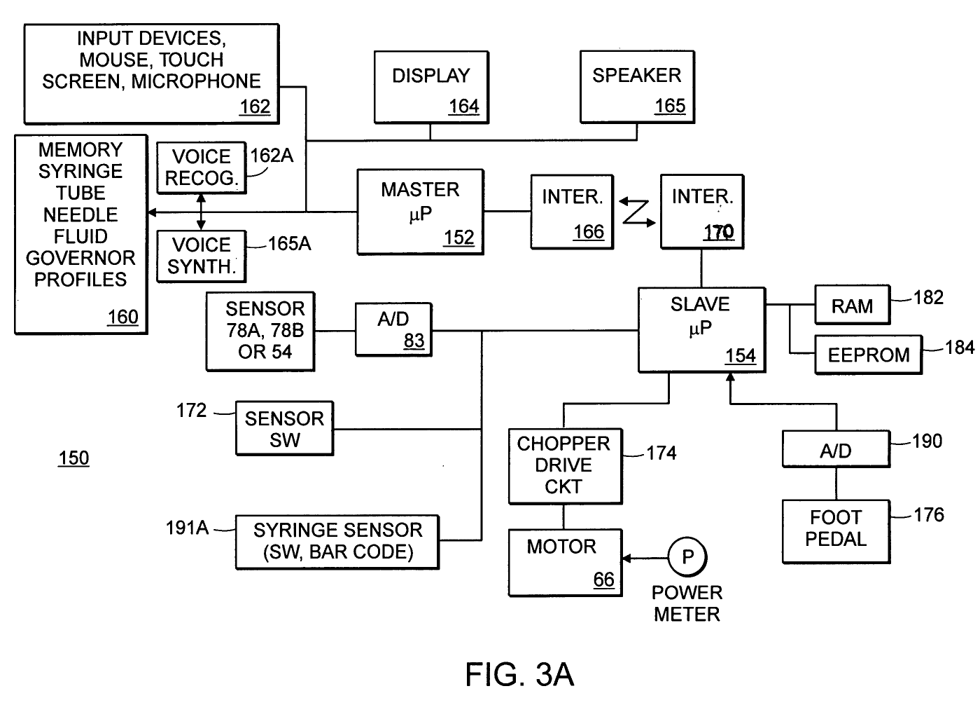

The mechanical assembly for the subject system is illustrated in FIGS. 1 and 2 and the electronic controller 150 for the system is shown in FIG. 3.

A drug delivery system 10 constructed in accordance with this invention includes drive mechanism 12, a delivery tube 14 and a handle 16 terminating with a needle 17. More particularly, a syringe 90 (or other fluid storage device) is mounted on the drive mechanism with one end of tube 14 being coupled to the syringe 90. The drive mechanism 12 operates a plunger 94 to selectively eject fluid out through the tube 14 handle 16, and needle 17 or alternatively to draw fluid in. The drive mechanism 12 is associated with an external controller for selecting various operational parameters discussed in more detail below. This external controller may be provided on the housing of the drive mechanism or may be provided as a separate control unit 18 coupled to the drive mechanism 12 by a cable 20. The control unit 18 may be for instance a PC or laptop computer. Alternatively, the control unit 18 may be internal.

Details of the drive mechanism 12 are seen in FIG. 2. The drive mechanism 12 includes a housing 22 with a top surface 24 and intermediate surface 26 disposed below top surface 24. On surface 26 there is formed a rail 28 extending along the longitudinal axis of housing 14. A platform 30 which is disposed on the rail 28 can be reciprocated back and forth in parallel with said longitudinal axis, as described in more detail below.

On top surface 24 there is a clamp 40. The clamp 40 has a generally C-shaped body. A screw with a head 48 extends through a threaded hole (not shown) in the body of the clamp 40. Platform 30 has a slot 56.

Inside the housing 22, there is provided a motor 66 (FIG. 3). Threaded through the motor 66 there is a worm screw 72. The worm screw 72 is arranged so that as the motor 66 is activated, the worm screw 72 moves in one direction or another, dependent on its direction of rotation, in parallel with the longitudinal aids of the housing 22. One end of the worm screw 72 is non-rotatably attached to a pad 74, coupled to a platform 76. Two short rods 80 are used to couple the pads 74 to platform 76, to prevent the transmission of rotational forces generated by the motor 66 to the platform 76.

Two columns or rods 82, 84 extend between platforms 30 and 76 and secure these two members together. These rods 82, 84 are slidably supported by two pairs of bushings 68, 70 on the housing 22. Except for these bushings, the platforms 76 and 30 are floating respectively inside and outside the housing 22. Rods 82, 84 extend through wall 86 extending between surfaces 24 and 26 via holes (not shown). The rail 28 is hollow and aligned with the worm screw 72 to allow the worm screw 72 to move longitudinally along its axis through the housing 22.

Typically, the syringe 90 has a barrel 92 on surface 24. The barrel 92 has a finger tab resting in a slot formed on the face 24. The finger tab and the slot have been omitted from the drawings for the sake of clarity. The syringe 90 also includes a plunger 94 reciprocated within the barrel 92 by a shaft 93. The shaft terminates in a finger pad 96 resting in slot 56 of platform 30. The syringe 90 is secured to the housing 22 by clamp 40 and screw 48. The syringe terminates with a Luer lock 95 used to connect the syringe to tube 14.

When the motor 66 is activated, as discussed below, it forces the worm screw 72 to move in one direction or another. The worm screw in turn forces the platforms 30, 76 and rods 82 and 84 to move in concert as well, thereby forcing the plunger 94 to reciprocate within the barrel 92. The only elements which move in and out of the housing are the rods 82, 84. Hence most of the critical elements of the system are protected within the housing from tampering, or spilled fluids. Moreover, the drive mechanism 12 is adapted to receive and operate with syringes of various diameters and lengths. Similarly, the delivery tube 14, handle 16 and needle 17 may have any size desired. More details of the syringe and the motor drive, the worm screw and its coupling to the platform 30, are described in U.S. Pat. No. 6,200,289. Moreover, this patent further describes a load cell 78 disposed between platform 76 and pad 74 and arranged to transmit and measure the force between the pad 74 and platform 76. This load cell 78 is bidirectional so that it can measure both stress and strain dependent on whether the worm screw 72 is moving to the left or to the right as determined in FIG. 3. In the present invention other means are disclosed that replace this load cell.

In one embodiment, the apparatus includes a pair of pressure sensors 78A are disposed between finger pad 96 and the walls of slot 56. The sensors 78A are arranged to measure the force applied between the platform 30 and the finger pad 96.

In another embodiment, sensors 78B are provided between the bushings 68 and the sidewalls of the housing 22. In this manner, the sensors 78B can measure the force (or strain) resultant from the force applied by the motor on the syringe plunger 94. Alternatively, a similar load cell may be placed between the syringe tab and the housing 22. The sensors may be load cells, for instance a Model S400 load cell made by the SMD, Inc. of Meridien, Conn.

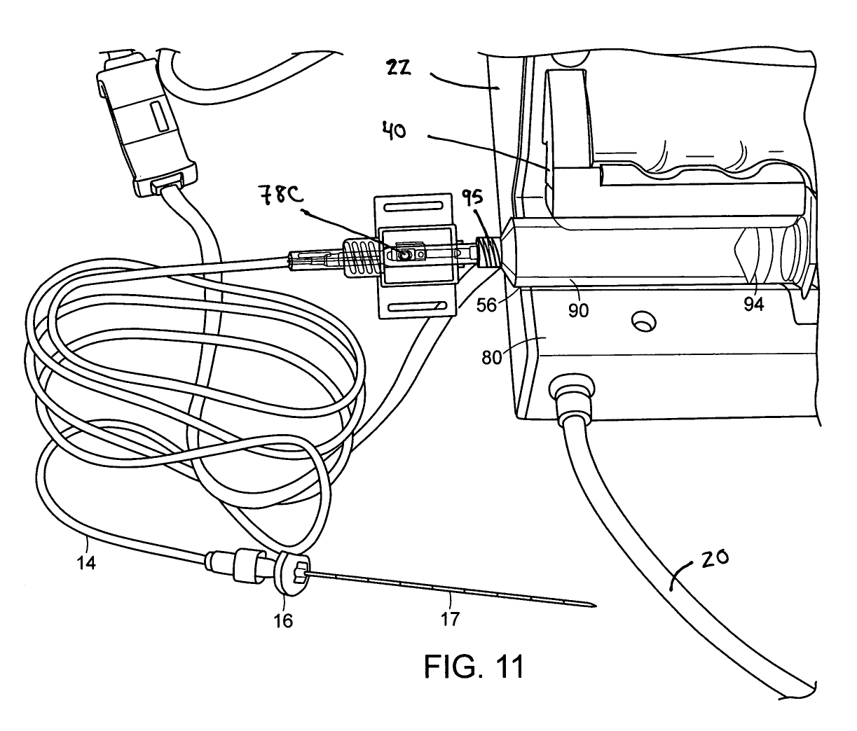

In another embodiment, an in-line fluid pressure sensor 78C is provided either between the syringe (fluid reservoir) 90 and the tubing 14, or between the tubing 14 and the needle 17. FIG. 11 shows on particular configuration of the injection apparatus in accordance with the principles of this disclosure. The in-line fluid pressure sensor 78C is disposed between the syringe 90 and the tubing 14. Omitted from FIG. 11 is the cable interface between the fluid pressure sensor 78C and the external controller.

In yet another embodiment, shown in FIG. 1, the tubing 14 passes through a hole in a size gauge 54. When the tubing 14 is pressurized, it expands, and therefore, the size of the tubing is indicative of the pressure applied thereto by the plunger. The size gauge 54 monitors the size (e.g. cross-sectional dimension, or diameter) of the tubing 14 and provides this parameter to the master controller 18. For example, gauge 54 may include one or more LEDs and an array of light sensors with tubing disposed therebetween. The size of the tubing is determined by the number and/or position of the light sensors occluded by the tubing.

FIG. 8 shows a cross-section of another gauge 54A that may be used instead of gauge 54. It consists of a base B with a slot S holding the tubing T. A hinged cover C holds the tubing T in place. A force sensor FS available off the shelf is inserted through a hole H and rests against the tubing T. As the tubing expands and contracts due to pressure changes, it applies a force on the force sensor. Experimental data shows that this gauge 54A has a fairly linear output and easy to calibrate for various pressures.

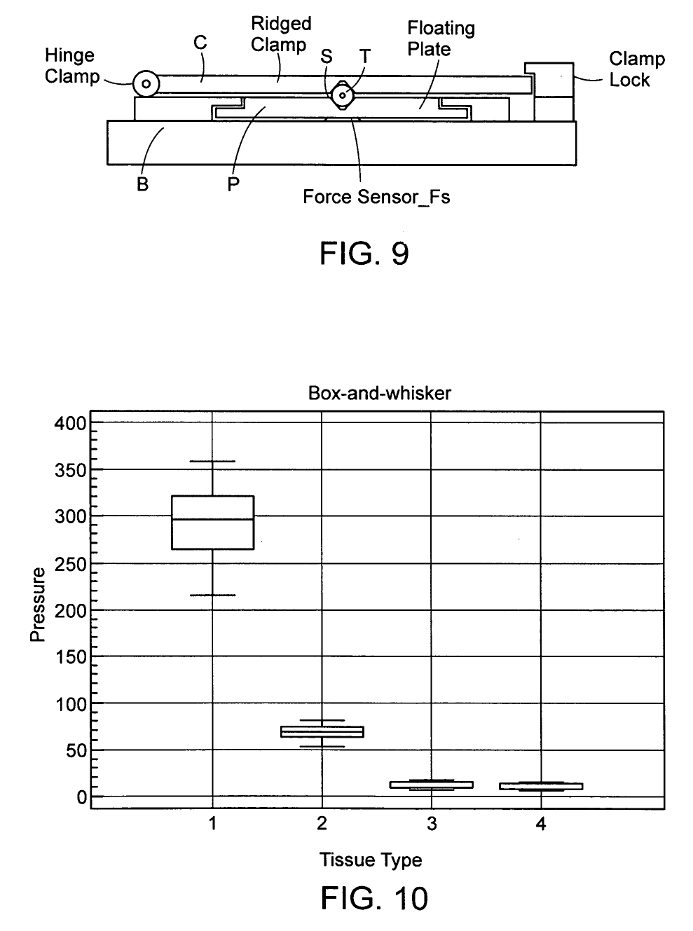

FIG. 9 shows another gauge 54B that can be used instead of gauge 54. This gauge is similar to the one in FIG. 8 with the exception that a groove is made in the cover C and the tube is resting on a floating platform P disposed above the force sensor. The force generated by the pressure within the tube is transmitted by the floating platform P to the force sensor FS. Again, the response of this gauge is linear and easy to calibrate.

FIG. 3A shows a block diagram of the electronic controller 150. The controller 150 includes two microprocessors: a master microprocessor 152 and a slave microprocessor 154. Slave microprocessor 154 is used to derive the signals that actually drive the motor 66 and to collect information regarding the position of the platforms 30, 76.

The master microprocessor 152 is used to collect information regarding the rest of the system, including the syringe 90, and its contents, the tube 14, the handle 16 and so on, and to generate control signals for the slave microprocessor 154 necessary for operating the motor 66 to deliver the contents of the syringe 90.

Physically, the slave microprocessor 154 and its associated circuitry are disposed within the housing 22. The master microprocessor 152 is incorporated into control unit 18 which is coupled to the housing 22 through cable 20 as shown in FIG. 1. The microprocessor 152 is associated with a memory 160, input devices 162, display devices 164 and an interface 164.

Memory 160 is used to store programming and data for the master microprocessor 152. More specifically, the memory 160 is used to store six or more data banks, each of said data banks being dedicated to the following information: (a) syringes; (b) tubing; c) needles; (d) fluids; (e) governor parameters; and (f) profiles consisting of a plurality of parameters for a particular procedure to be performed. Each of these parameters is used to determine the control signals generated for the slave microprocessor 154. Each of these data banks contains the appropriate parameters for various commercially available products, or alternatively, parameter data derived using a specific algorithm. Information regarding the various elements for a particular configuration is entered through input devices 102 and is confirmed on the display device 164. These input devices may include a keyboard, a touch screen, a mouse, as well as a microphone. If a microphone is included, voice commands are interpreted by a voice recognition circuit 162A.

The display device 164 is further used to provide an indication as well as instructions on the operation of the system 10. The commands for the operation of motor 66 are generated by master microprocessor 152 and transmitted to an interface 162. Microprocessor 152 is further provided with a speaker 165 used to provide various oral messages, including spoken pre-recorded or synthesized words, (generated by a voice synthesized circuit 165A) chimes, and so on, to provide instructions to the clinician and to provide other information about the current status of the whole system and its elements without the need for the clinician to look at the displays all the time.

The slave microprocessor 154 receives these commands through cable 20 or other connection means and interface 170.

Also associated with the slave microprocessor 154 are one or more position sensors 172 and a chopper drive circuit 174. As previously mentioned, the force or pressure generated within the system is measured by sensors 78A, 78B, 78C, 54, 54A, 54B.

Also associated with slave microprocessor 154 is a foot switch or pedal 176. Preferably foot pedal 176 consists of an air chamber with a flexible side wall, said side wall being arranged to change the volume of air and pressure within said chamber in response to activation by a human operator. A pressure sensor (not shown) is part of the foot pedal and is arranged to provide information about said pressure to slave microprocessor 154 via a corresponding A/D converter 190. Foot pedals of this kind are well known in the art and therefore its details have been omitted.

The sequence of operation for the system 10 are similar to the ones described in U.S. Pat. No. 6,200,289 (hereby incorporated by reference) and are not repeated here. Moreover, the algorithm disclosed in said patent is also applicable for converting the parameter obtained from the sensors 78A, 78B, 78C, or 54 into a corresponding exit pressure.

In another embodiment, the power required to drive motor 66 is monitored. For example, the master controller 150 maybe provided with a power meter P that monitors this power, for example, by measuring the voltage and current applied thereto. This power is, of course, indicative of the force applied by the motor and is used in the same manner as the output of the sensors 78A, 78B or 54.

The system has been described so far as performing an injection process. However, it is obvious to one skilled in the art that it can be used just as effectively to perform a biopsy, for instance to perform a spinal tap, or other similar anaerobic procedures. Essentially the same parameters can be used for this process, with some minor modifications. For instance, instead of defining an exit pressure, the clinician now defines an entry pressure.

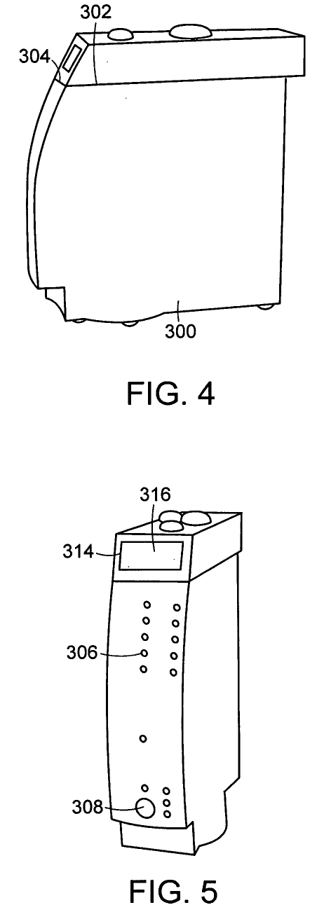

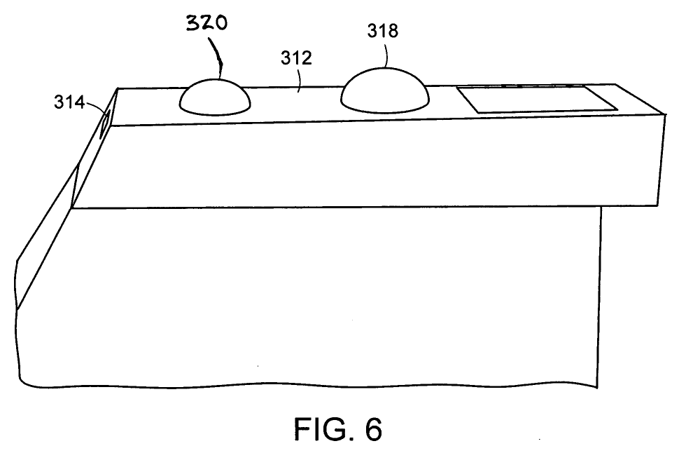

In the embodiment discussed so far, it is assumed that a fluid is dispensed from the syringe 90 and, therefore, this syringe 90 must be preloaded with said fluid either by the manufacturer, or must be filled at the site by the clinician or an assistant prior to the start of any operation. In many procedures, however it is more desirable to provide the fluid to be dispensed in a cartridge. Commonly owned U.S. Pat. No. 6,152,734 an injection device is described that includes a housing with a motor driven shaft. On top of the housing, a receptacle is provided for accepting a cartridge holder. The cartridge holder receives a cartridge with an anesthetic. The holder has a top wall connected to the proximal end of a tubing. The distal end of the tubing is used to deliver an anesthetic through its distal end. In accordance with this invention, a sensor module is added on top of the housing. Referring first to FIGS. 4, 5 and 6, the housing 300 has a top surface 302 and a front surface 304. Disposed on the front surface 304 there are a plurality of indication lights and one or more control buttons 308. According to this invention, a sensor module 310 is mounted on top surface 302. This module 310 includes its own upper surface 312 and front surface 314. On front surface 314 there is an LCD display 316.

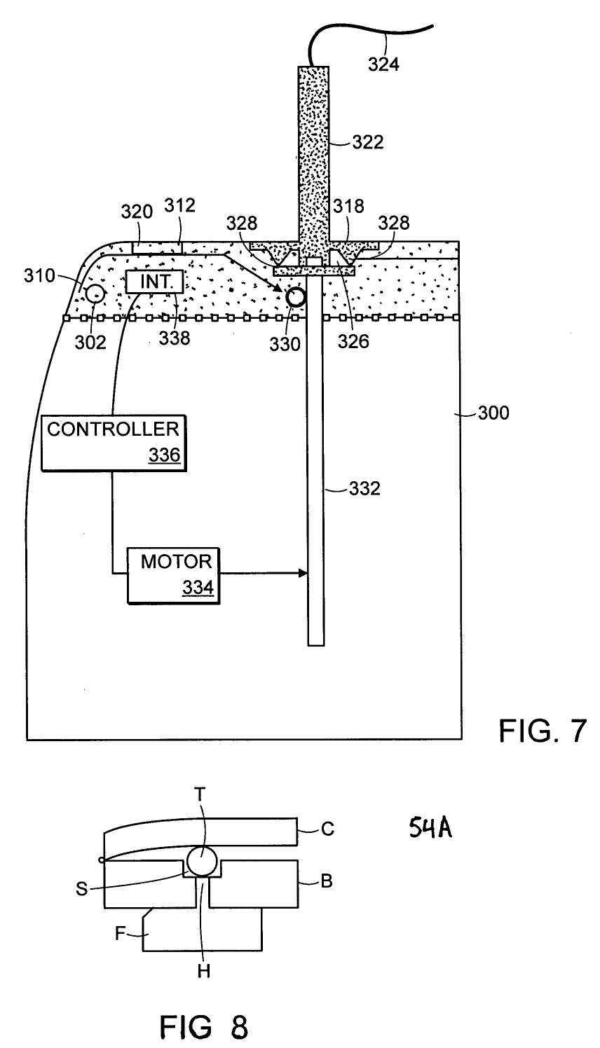

On the top surface 312 there is provided a receptacle 318 and a hole 320 having the same shape and size as the corresponding elements on the top of the housing 300 described and illustrated in U.S. Pat. No. 6,152,734. Referring now to FIG. 7, attached to module 310 there is a cartridge 322 connected to the proximal end of a tubing 324. The distal end of the tubing is connected to a syringe, a catheter or other similar injection means (not shown). When not in use, this injection means can be stored in hole 320. The bottom 326 of the cartridge holder 322 is shaped so that it can be inserted quickly and easily into the receptacle 318 and form an interference fit therewith. As described in U.S. Pat. No. 6,152,734, preferably a quick-connect coupling is provided between the bottom 328 and the receptacle 318 so that the cartridge holder 322 can be quickly and easily installed onto and removed from the receptacle. The cartridge holder 322 holds a cartridge with an anesthetic or other medicinal substance (not shown).

Importantly, according to this invention, one or more sensors 328 are positioned between the bottom 326 of cartridge holder and the walls of receptacle 318. These sensors may be pressure sensors or other similar sensors used to monitor the force applied to the liquid being ejected through tubing 324.

As discussed above, disposed in housing 300 there is a plunger 332. Module 312 holds optionally a plunger sensor 330 that is disposed in close proximity to, or in contact with the plunger 332. As the plunger moves upward, its tip enters into the cartridge in the cartridge holder 322 and forces its contents to be ejected through tubing 324. Moving plunger 332 downwardly causes aspiration. The plunger sensor 330 measures the direction and, optionally, the rate of movement of the plunger 332.

This plunger 332 is reciprocated vertically by a motor 334. The motor 334 is controlled by a controller 336. The sensors 328 and 330 are coupled to an interface 338. This interface transmits the information from the sensors 348, 330 to the controller 336. The controller then operates the motor to cause the plunger 332 in the same manner, and using the same algorithm as the plunger 94 in FIGS. 1-4. The information associated with this operation, and any other information are displayed on the display 316.

In this arrangement the sensors may also be used to detect basic operations of the unit such as purging or auto-retraction of this plunger. As the cartridge holder is inserted within the socket of the drive unit the pressure sensors detect their placement and will then automatically purge air from the tubing line readying the system for use. When the cartridge holder is removed from the unit the pressure sensor can detect the removal and allow for automatic retraction of the plunger to the “home” position. Hence, the pressure sensors play a multipurpose role of detecting exit pressure as well as basic operations of the drive unit.

Importantly, the pressure may also be used as criteria to determine the tissue in which fluid is being injected by the device. Previous authors have investigated the clinical implications of interstitial pressure during dental injections. The present inventor has conducted research that demonstrates that using the device described herein, subcutaneous interstitial pressures could be accurately measured and recorded in real-time. It was also determined that a given range of pressures obtained with the device could be readily identified and associated with for specific tissue types. Interstitial pressures generated were correlated to the tissue densities type for particular anatomic locations.

Highly organized densely packed collagen fibers such as those found in certain oral tissues as in the periodontal ligament and gingival hard palate reduced the ability for diffusion of injected fluid, i.e. fluids are contained within a smaller area. This reduced ability for denser tissues to allow rapid re-distribution of the drug results in higher internal pressure during injections. In contrast, loosely organized tissues with a connective stroma composed of a collagen matrix interposed with interstitial fluid and adipose tissues as those found in the mucobuccal fold and infratemperal fossa, result in lower interstitial pressure, as a result of the drug being spread through a larger tissue area.

From this observation, a conjecture was made that there was a correlation between tissue density type and the injection process. More specifically, tests were conducted on the following types of injections:

Group 1—intraligamentary injections (PDL) (a.k.a. periodontal ligament injection), Group 2—the anterior middle superior alveolar palatal injection s(PI), Group 3—the supra-periosteal buccal infiltration (SBI) and Group 4—the inferior alveolar nerve block (IANB). FIG. 10 shows the various pressures obtained during these injections, and clearly illustrates the concept of using pressure (preferably exit pressure) as a means of identifying tissues.

In general, tissues may be categorized into the following types:

Type 1—Low density tissues, comprised of a loosely organized connective tissue matrix interposed with adipose tissue, intercellular fluids and small volumes of organized collagen fibers present. Examples of this tissue type are subcutaneous connective tissues of the maxillary buccal mucosa and infratermporal fossa. Examples of injections performed in these tissue types include; buccal infiltration and inferior alveolar nerve block.

Type 2—Moderate density tissues, comprised of a combination of densely packed collagen fiber bundles interposed with a small amount of glandular tissues and/or adipose tissue. A relatively small amount of intercellular fluids are found in these tissues. Moderate density tissues would also be represented by muscular tissue of the oral cavity. A moderate degree of collagen organization is found in these types of tissues. The tissue types are represented by the attached palatal gingiva, attached gingival tissues or muscle tissues of the oral cavity. Examples of injections performed in these tissue types include; palatal injections or injections into the attached gingiva.

Type 3—High density tissue composed of predominately dense highly organized collagen fiber matrixes. Examples of these types of tissues are the periodontal ligament and the muscle tendon attachments, an example of an injection performed in this tissue type is the PDL injection.

Moreover, the use of these techniques can be expanded to identify both mineralized and non-mineralized tissues, and even fluids as follows:

Non-mineralized tissues:

Soft tissues, connective tissues, dermis (skin),

Ligaments

Adipose tissues (fat)

Muscle

Tendons

Brain tissues

Vessels

Mineralized tissues:

Cortical Bone

Medullary bone

Cartilage

Teeth

Neoplasms:

Hard and soft lesions

Fluid filled lesions

Hematomas

Cysts

Fluids: Extacellular and Intra-cellular Fluids

Intra-capsular fluids of Joints

Intra-cranial fluids

Cerebral Spinal fluids

Lymph fluid

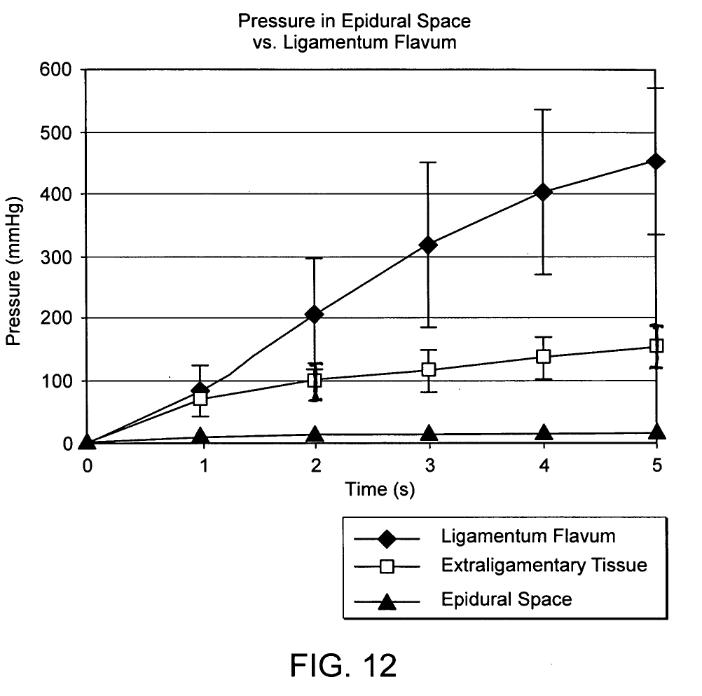

The proper identification of the epidural space is crucial for efficient and safe epidural anesthesia. Thus, it is necessary to characterize the surrounding tissues through which the injection needle must pass in terms of the expected exit pressure. The exit pressure associated with the ligamentum flavum was measured in twenty obstetric patients. An injection device configured in accordance with the principles of this disclosure was attached to a Tuohy epidural needle and the needle was introduced to a depth of 3 cm to measure the baseline exit pressure, then advanced to the ligamentum flavum, and advanced again to the epidural space. The latter identified by the loss-of-resistance. Pressure measurements were recorded for 5 sec at each location. FIG. 12 is a graph of the calculated exit pressure in the epidural space (triangles) which is significantly less than the exit pressure of the extraligamentary tissue (squares), which itself is significantly less than that of the ligamentum flavum (diamonds).

As described above, the injection device continuously monitors a pressure, and preferably the exit pressure of the fluid during injection. Based on tables stored in its memory, the device is able to determine the type of tissues in which the injection is being injected. This information is displayed to the doctor (or other clinician). The doctor can then confirm that he is performing the injection in the desired tissues. In addition, for each type of tissue, preselected maximum allowable pressure limits and/or flow rates are stored that define either the maximum recommended pressures that patients usually tolerate, or other criteria. The parameters are stored in memory 160. As the pressure approaches this limit, a visual and/or audible alarm is generated for the clinician. In addition, data descriptive of the whole injection process is stored for future analysis, as discussed above.

Method for Administering an Epidural Injection

An exemplary method for administering an epidural injection follows. It is contemplated that the exact settings and tolerances will be modified by the clinician. These principles and methods may be easily adapted for injections into tissues and anatomical areas other than the epidural space.

The upper exit pressure limit is determined by the clinician. Typically, the upper exit pressure limit to not greater than 100 mm/Hg. It is contemplated that, using such a setting, the injection system will administer a negligible amount of medication unless the needle is properly positioned within the epidural tissue-space because the pressure within the epidural tissue space is believed to be between about +15 mm/Hg and −15 mm/Hg, whereas the exit pressure associated with the Ligamentum Flavum is above 200 mm/Hg. Exit pressure measurements within the extra-ligamentary tissues are typically about 100-200 mm/Hg. With the injection device having a pre-set maximum exit pressure that is 100 mm/Hg or below, there will be no significant fluid flow once the needle enters the subcutaneous tissues as the exit pressure will quickly rise and be maintained as long as the needle resides within the subcutaneous tissues (extra-ligamentary tissues). The clinical, following traditional epidural injection technique, will advance the Touhly needle and encounter the ligamentum flavum. Still no fluid flow will occur because, as noted above, the ligamentum flavum generates an exit pressure greater than 100 mm/Hg. Upon penetrating the ligamentum flavum (i.e., needle entry into the epidural space) the exit pressure will immediate drop below 100 mm/Hg triggering an optional visual display and/or audible tone, and the drug-containing fluid will begin to flow.

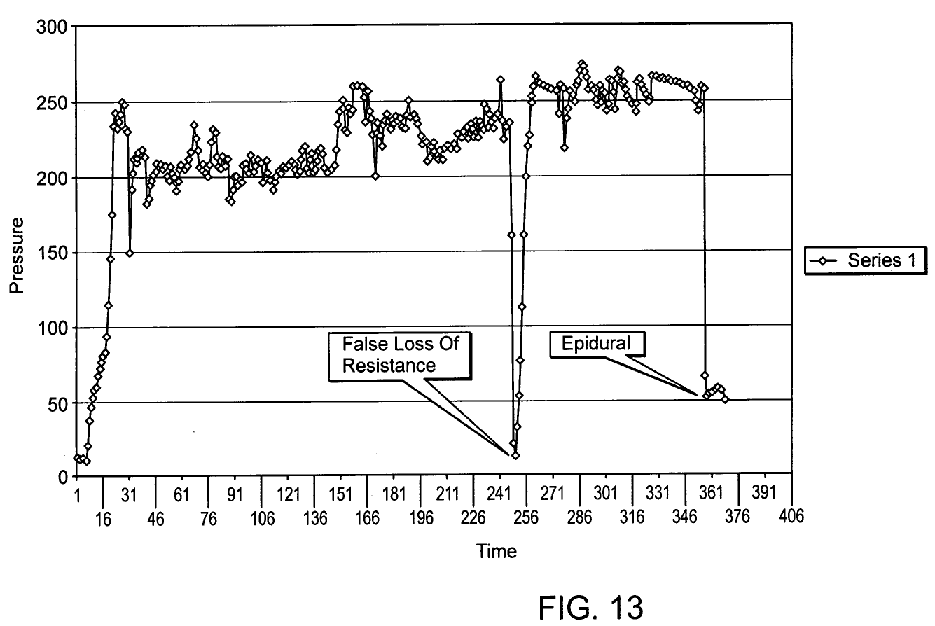

The pressure sensors of the injection device provide an automatic safety feature in the event that the injection needle leaves the epidural tissue space (e.g., from clinician error or patient movement) or its patency is compromised. If the needle leaves the epidural tissue-space, either by withdrawing through the ligamentum flavum or by contacting the dura, the exit pressure will immediately rise causing a slowing and eventual stoppage of fluid flow at exit pressures >100 MM/Hg. This has been shown to occur within approximately 2 seconds time (see, FIG. 13). Optionally, this change in exit pressure from <100 mm/Hg to >100 mm/Hg will again trigger a visual and/or audible alarm to alert the clinician of improper needle placement. Flow will again automatically resume once the needle is reestablished in the epidural tissue space. This automatic safety feature of the injection device helps prevent injection of the anesthetic solution into the spinal cord.

A feature of the present injection device and accompanying method is the ability to quickly and accurately identify a “false-loss-of-resistance” or “false-positive” (typically within 2-4 seconds). A false-loss-of-resistance typically occurs when a traditional loss-of-resistance manual syringe technique is used and a drop of resistance occur when the epidural needle enters a cyst or less dense space outside the epidural tissue-space. The ligaments in the area are understood to be less dense and a false loss of resistance is not uncommon. Many times the subjective nature of this anatomic location can lead the clinician to believe he has located the epidural tissue-space. When using the computer-controlled drug delivery system with exit pressure control once the needle enter such as space it would quickly fill the space or pressurize the less dense tissue with fluid and the recorded exit pressure would once again raise above 100 mm/Hg and objectively indicate a “false-loss-of-resistance”. This would typically not be the situation using a traditional manual syringe technique as once the initial loss-of-resistance is encountered the syringe is removed and the operator delivery the bolus of the fluid no longer subjectively testing for a “loss-of-resistance” thereby depositing anesthetic solution in an anatomic location outside the intended epidural tissue-space. FIG. 13 is a line graph demonstrating a false-loss-of-resistance (at time ˜250 sec.), associated most likely with ligamentous tissue, measured during the administration of an epidural injection. The incorrect tissue structure was quickly pressurized, returning the measured exit pressure >200 mm/Hg. Insertion of the catheter into the epidural space and subsequent fluid injection does not result in a significant and rapid rise in exit pressure, indicating that the catheter is correctly located.

It is contemplated that a pharmaceutical-free fluid is used to identify the epidural tissue space during the needle placement phase of the epidural procedure. Suitable pharmaceutical-free fluids include, for example, sterile saline, artificial cerebral spinal fluid, Ringers, 5% dextrose, or filtered air. Once the epidural tissue space is identified using the loss-of-resistance method, the injection fluid is changed to a pharmaceutical-containing fluid. The use of a pharmaceutical-free fluid during the needle placement phase minimizes or eliminates the delivery of the pharmaceutical to non-target tissues.

Another feature of the current device and methodology is the objective nature of exit pressure measured by a computer-controlled drug delivery device that is continuously monitored during all phases of the injection process. The clinician, therefore, no longer relies on the subjective nature of a “feel” but rather is provided with objective information of absolute values will performing each phase of this critical technique. Each phase of the technique is improved by the ability to continuously monitor the exit pressure allowing adjustments to be made that ensure greater safety and efficacy of the injection.

In another example, the clinician may reset the maximum allowable exit pressure once the epidural space is penetrated and the injection has begun. As noted above, prior to needle entry into the epidural space, the fluid exit pressure is greater than 100 mm/Hg so little or no fluid is being delivered. Upon entry of the epidural space the exit pressure drops below zero and gradually rises to about 1-10 mm/Hg. This drop in exit pressure initiates the flow of fluid from the injection device. At this time, the maximum pre-set exit pressure value may be changed to a new, lower, maximum. For example, the maximum exit pressure may be reduced to 25 mm/Hg which will provide an extra level of patient safety in the event that the injection needle contact or puncture the dura or is withdrawn from the epidural space. The lower maximum exit pressure will cause the fluid flow to be arrested sooner, and at lower ectopic injection amounts, than the original pre-set value. The change in maximum exit pressure may be performed manually by the clinician or automatically by a control element in the injection device. In the latter case, the control element may be programmed to trigger the parameter change once a particular exit pressure (e.g., 10 mm/Hg) is obtained.

Injection devices and methodologies consistent with the principles of this disclosure are capable of accurately accounting for a change in the components of the drug delivery system including, for example, changes in syringe size, tubing bore and length, needle bore and length, and solution viscosity. The current device provides a means to input changes and recalculate the effect it will have on the absolute value measurement of exit pressure. This is a critical element to the systems accuracy and flexibility. For example, the clinician may shorten a tubing component, such as an indwelling catheter tubing, by cutting a piece from the original tubing set that has a defined length and measured resistance properties. The tubing fragment is measured and the discarded length is inputted into the injection device software system which calculates the exit pressure. Using the changed information, the software system is able to recalculate the actual exit pressure in the modified tubing system. Likewise, the injection device software system accounts for variables including injection fluid viscosity and temperature, etc.; each of which may be changed and affect the calculated exit pressure.

It should be understood that the example of 100 mm/Hg as the maximum pre-set exit pressure is an example and that either a lower or higher pre-set exit pressure may be selected at the discretion of the clinician. Also, the example of an epidural injection is merely illustrative. The principles and techniques may be modified for an injection into almost any anatomical location. What is of particular importance in the method and device for this embodiment is the ability to define and select absolute values of exit pressure when utilizing this device and method.

The techniques described herein are equally applicable to human and animal tissues.

While the invention has been described with reference to several particular embodiments, it is to be understood that these embodiments are merely illustrative of the principles of the invention. Accordingly, the embodiments described in particular should be considered as exemplary, not limiting, with respect to the following claims.

What is claimed is:

1. An apparatus comprising:
a fluid reservoir,
an injection fluid,
a pumping mechanism capable of pumping said injection fluid at a flow rate,
an end adapted to be inserted into the body of a patient, wherein said end is in fluid contact with said fluid reservoir;
a sensor arranged to determine a measurement, and
a controller capable of receiving said measurement, calculating the exit pressure of said injection fluid, and modulating said flow rate.

2. The apparatus of claim 1, wherein said controller is further capable of receiving user-inputted parameters.

3. The apparatus of claim 2, wherein said user-inputted parameters comprise a maximum exit pressure value.

4. The apparatus of claim 3, wherein said controller is programmed to reduce said flow rate to zero when said exit pressure measurement exceeds said maximum exit pressure value.

5. The apparatus of claim 3, wherein said maximum exit pressure value is between about 50 mm/Hg and about 300 mm/Hg.

6. The apparatus of claim 5, wherein said maximum exit pressure value is between about 150 mm/Hg and about 250 mm/Hg.

7. The apparatus of claim 2, wherein said user-inputted parameters is at least one parameter selected from the group consisting of injection tubing length, injection tubing bore, injection fluid viscosity, injection fluid composition, and injection fluid temperature.

8. The apparatus of claim 1, wherein said sensor is an in-line pressure sensor disposed between said pumping mechanism and said end, and wherein, said measurement is the fluid pressure of said injection fluid.

9. The apparatus of claim 1, wherein said sensor is attached to said pumping mechanism.

10. The apparatus of claim 1, wherein said end is adapted for insertion into the epidural tissue space.

11. The apparatus of claim 10, wherein said end is a needle or a catheter.

12. The apparatus of claim 1, wherein said injection fluid comprises an anesthetic.

13. The apparatus of claim 4, wherein said apparatus further comprises a visual signal, under the control of said controller, wherein said visual signal indicates each time said flow rate starts or stops.

14. The apparatus of claim 4, wherein said apparatus further comprises an audible signal, under the control of said controller, wherein said audible signal indicates each time said flow rate starts or stops.

15. A method for administering an injection to a patient, said method comprising:
providing a fluid reservoir, an injection fluid, a pumping mechanism, and an end adapted for insertion into said patient,
pumping said fluid from said reservoir into said patient,
calculating the exit pressure of said fluid at an interface between said end and the tissue of said patient, and
controlling the flow rate of said injection fluid such that said exit pressure does not exceed a pre-set maximum exit pressure.

16. The method of claim 15, wherein a measurement is obtained using a sensor and said exit pressure is calculated using said measurement.

17. The method of claim 16, wherein said sensor is an in-line pressure sensor disposed between said pumping mechanism.

18. The method of claim 16, wherein said sensor is functionally coupled to said pumping mechanism.

19. The method of claim 15, wherein said flow rate is substantially zero when the measured exit pressure is equal to or greater than the pre-set maximum exit pressure.

20. The method of claim 15, wherein said flow rate does not exceed a pre-set maximum flow rate.

21. The method of claim 15, wherein said flow rate is a function of said exit pressure.

22. The method of claim 15, wherein said injection is made into the epidural tissue space.

23. The method of claim 15, wherein said pre-set maximum exit pressure is between about 50 mm/Hg and about 300 mm/Hg.

24. The method of claim 23, wherein said pre-set maximum exit pressure is between about 150 mm/Hg and about 250 mm/Hg.

25. The method of claim 15, wherein said injection fluid comprises an anesthetic.

26. The method of claim 22, wherein said method further comprises the step of testing said exit pressure by aspiration prior to injection of said injection fluid in order to identify said epidural tissue space.

27. The method of claim 22, wherein said method further comprises the steps of:
administering a second injection to said patient,
calculating the exit pressure of said fluid at an interface between said end and the tissue of said patient during said second injection, and
controlling the flow rate of said injection fluid during said second injection such that said exit pressure does not exceed said pre-set maximum exit pressure.

28. A method for administering an epidural injection into the epidural tissue space, said method comprising:
providing a testing fluid, an injection fluid, a pumping mechanism, and an end adapted for insertion into said patient,
pumping said testing fluid into said patient,
calculating the exit pressure of said testing fluid at an interface between said end and the tissue of said patient,
controlling the flow rate of said injection fluid such that said exit pressure does not exceed a pre-set maximum exit pressure,
identifying the loss-of-pressure associated with the entry of said end into said epidural tissue space, and
pumping said injection fluid into said epidural tissue space.

29. The method of claim 28, wherein said testing fluid is selected from the group consisting of physiological saline, phosphate-buffered saline, artificial cerebral spinal fluid, Ringers, 5% dextrose, and filtered air.

30. The method of claim 28, wherein said injection fluid comprises a therapeutic agent.

31. The method of claim 30, wherein said therapeutic agent is an anesthetic.
